(19) United States
(12) Plant Patent Application Publication
(10) Pub. No.: US 2013/0239281 P1
(43) Pub. Date: Sep. 12, 2013

(54) Kniphofia plant named 'Lemon Popsicle'

(50) ;

(71) Applicant: Janet N. Egger, Wilsonville, OR (US)

(72) Inventor: Janet N. Egger, Wilsonville, OR (US)

(73) Assignee: Terra Nova Nurseries, Inc.

(21) Appl. No.: 13/385,799

(22) Filed: Mar. 6, 2012

(51) Int. Cl. A01H 5/00

(52) U.S. Cl. PLT/443

(57) ABSTRACT

A new and distinct Kniphofia plant characterized by numerous spikes of yellow flowers the first year, repeat blooming from June through September in Canby, Oreg., a compact habit with multiple crowns, narrow, grassy leaves, and excellent vigor.

### BOTANICAL DENOMINATION

Kniphofia spp.

### CULTIVAR DESIGNATION

Lemon Popsicle

### BACKGROUND OF THE INVENTION

The present invention relates to a new and distinct Kniphofia, and given the cultivar name is ‘Lemon Popsicle’. Kniphofia is in the family Asphodelaceae. ‘Lemon Popsicle’ is a hybrid resulting from a planned breeding program to produce a series of compact, reblooming Kniphofia. The cross was made using the following proprietary, unreleased plants: Kniphofia 8-1, as the seed parent, and Kniphofia 9-1, as the pollen parent. It was selected for best habit, flower color, reblooming, and crown count from many seedlings of the cross in Canby, Oreg.

Compared to Kniphofia ‘Little Maid’ (an unpatented plant), the new cultivar is more vigorous and has clear deep yellow flowers rather than bicolor light yellow and cream flowers.

Compared to Kniphofia ‘Dwarf Yellow’ (an unpatented plant), the new cultivar is a deeper yellow color and more free flowering.

### SUMMARY OF THE INVENTION

The new cultivar is unique and characterized by:
1. numerous spikes of yellow flowers the first year
2. repeat blooming from June through September in Canby, Oreg.
3. compact habit with multiple crowns
4. narrow, grassy leaves
5. excellent vigor, fills a one gallon pot the first year from tissue culture

This new cultivar has been reproduced only by asexual propagation (division, tissue culture). Each of the progeny exhibits identical characteristics to the new cultivar. Asexual propagation by leaf cuttings, tissue culture, and division using standard techniques as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

FIG. 1 shows a plant of a one-year-old Kniphofia ‘Lemon Popsicle’ in the ground in the garden in July in Canby, Oreg.

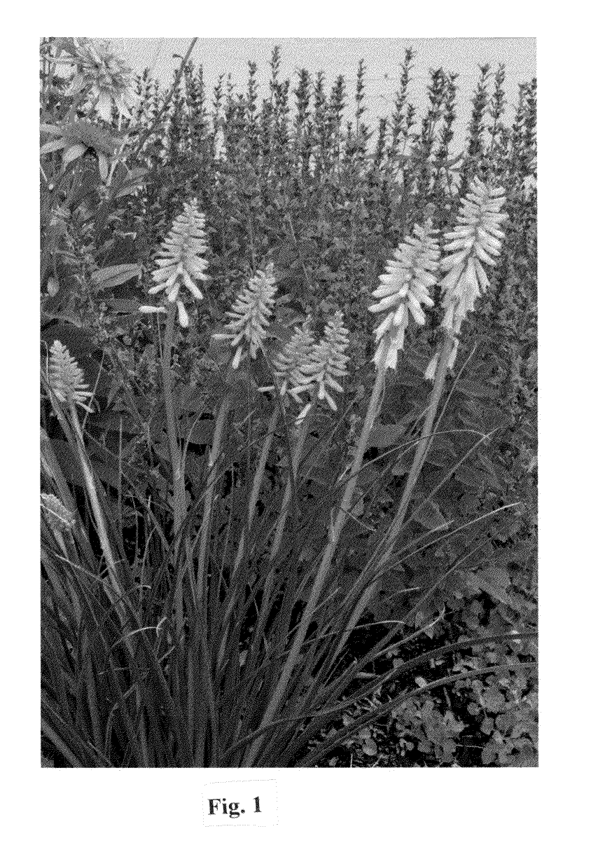

### DETAILED PLANT DESCRIPTION

The following is a detailed description of the new Kniphofia cultivar based on observations of one-and-a-half-year-old specimens growing in the ground outside in full sun in Canby, Oreg. Canby is in Zone 8 on the USDA Hardiness map. Temperatures range from a high of 95 degrees F. in August to an average of 32 degrees F. in January. Normal rainfall in Canby is 42.8 inches per year in the trial fields in Canby, Oreg. The color descriptions are all based on The Royal Horticultural Society Colour Chart, 5^{th} edition.
Plant:
   Type.—rhizomatous herbaceous perennial.
   Hardiness.—USDA Zones 6 to 9.
   Size.—grows to 45 cm wide and 45 cm tall from the top of the soil to the top of the foliage.
   Form.—clumping.
Leaf:
   Type.—simple.
   Shape.—strap-like.
   Arrangement.—basal rosette.
   Blade size.—grows to 50 cm long and 18 mm wide.
   Margins.—minutely serrulate.
   Apex.—acuminate.
   Base.—clasping.
   Surface texture.—glabrous on both surfaces.
   Venation.—parallel.
   Color.—top and bottom side Green 137A.
Inflorescence:
   Type.—spike-like scapose raceme.
   Number of flowers per raceme.—about 90.
   Inflorescence.—grows to 20 cm long and 4.4 cm wide.
   Peduncle description.—grows to 45 cm long and 7 mm wide, glabrous, Yellow Green 146B.
   Pedicle description.—2.5 mm long, glabrous, Yellow Green 146C.
   Bloom time.—late June through September in Canby, Oreg.
   Lastingness.—an inflorescence lasts for 2 to 4 weeks depending on the temperatures.
Flower bud:
   Size.—22 mm long and 5 mm wide.
   Shape.—cylindrical.
   Surface texture.—glabrous.
   Color.—Yellow 10B with tints of Green Yellow 1C near base and on veins.
Flower:
   Type.—actinomorphic.
   Shape.—cylindrical and tubular.
   Size.—grows to 2.8 cm deep and 10 mm wide.
   Texture.—waxy.
   Surface texture.—glabrous inside and outside.
   Color.—inside and outside, Yellow 5C darkening to 9C with veins Green Yellow 1C.
   Corolla description.—6 lobed fused tepals, 22 mm long and 11 mm wide, each lobe 3 mm wide and 4 mm long, ovate to oblong, margin entire, tip notched; glabrous inside and out.
   Pistil description.—one, 27 mm long, ovary 4 mm long 2.5 mm wide, Yellow Green 144A, style extruding, 18 mm long, stigma and style Green Yellow 1C.
   Stamen.—6, 31 mm long, filaments 30 mm long, and Green Yellow 1C, anthers 1.5 mm long and Yellow Orange 20A, pollen none.
   Fragrance.—none.
Fruit and seed: Few capsules seen, 5 mm long and 4 mm wide, glabrous, subglobous, Green 137A
Pest and diseases: No known resistances to pests or diseases. No problems have been observed on this plant grown under commercial conditions in Canby, Oreg.

I claim:

1. A new and distinct Kniphofia plant as herein shown and described.
